(19) United States
(12) Patent Application Publication
(10) Pub. No.: US 2020/0164078 A1
(43) Pub. Date: May 28, 2020

(54) APPLICABLE CHEMICAL COMPOSITION COMPRISING AN AGENT CONJUGATED TO A HYDROPHOBIC MOIETY AND A CARRIER

(71) Applicant: TECHNISCHE UNIVERSITEIT EINDHOVEN, EINDHOVEN (NL)

(72) Inventors: Patricia Yvonne Wilhelmina DANKERS, EINDHOVEN (NL); Maarten Herman BAKKER, EINDHOVEN (NL)

(21) Appl. No.: 16/632,271

(22) PCT Filed: Jul. 17, 2018
(86) PCT No.: PCT/EP2018/069393

§ 371 Date: Jan. 17, 2020

(30) Foreign Application Priority Data
Jul. 17, 2017 (EP) 17181727.3

(51) Int. Cl. A61K 47/54; A61K 47/69; A61K 9/00; A61K 31/407; A61K 47/10; A61K 47/60

(52) U.S. Cl. CPC A61K 47/554; A61K 47/6903; A61K 47/60; A61K 31/407; A61K 47/10; A61K 9/0019

(57) ABSTRACT

An applicable chemical composition is provided containing an agent with a hydrophobic functional group, e.g. a steroid, a sterol or in particular cholesterol, a derivative thereof, or any other chemical group that allows hydrophobic interactions, and possibly contains, but is not limited to, an additional OEG motif. The modified compound may be used alone or in combination with a carrier system, for example a hydrogel, for sustained release of the compound in the human body or in an aqueous environment. Advantageously, this combinatory compound-delivery system increases the therapeutic window for many drug molecules and requires a lower effective dose to induce the same therapeutic effect.

### FIELD OF THE INVENTION

The present invention lies in the field of controlled retention and release of agents, for example pharmaceutical drugs and other compounds that are administrated to patients. Indeed the possibility of sustained localized drug administration is of interest to physicians that are forced to treat patients with (exorbitantly) high systemic doses of drugs in order to obtain an local effective concentration. The invention also relates to compositions for controlled retention and/or release of such drugs or agents, and to methods directed to modifying retention and/or release of such drugs or agents. Also provided is for the medical use of such compositions, and for methods of treatment of patients wherein the treatment includes the use of the compositions as taught herein.

### BACKGROUND TO THE INVENTION

The field of local drug delivery, for example via hydrogels, is large, but the vast majority of prior art is described in scientific publications and holds no commercial follow-up. For example, hydrogels and their use in drug delivery are extensively described e.g. in Polymer, Volume 49, Issue 8, 15 Apr. 2008, Pages 1993-2007, ‘Hydrogels in drug delivery: Progress and challenges’. In Journal of Drug Delivery, Volume 2012 (2012), Article ID 103973, 17 pages, poly (ethylene glycol)-prodrug conjugates and applications have been described and poly (ethylene glycol) (PEG) is discussed. The following paragraph from this article has been cited hereunder.

‘Poly(ethylene glycol) (PEG) is the most widely used polymer in delivering anticancer drugs clinically. PEGylation (i.e., the covalent attachment of PEG) of peptides proteins, drugs, and bioactives is known to enhance the aqueous solubility of hydrophobic drugs, prolong circulation time, minimize nonspecific uptake, and achieve specific tumor targetability through the enhanced permeability and retention effect.’

Further there is prior art in the form of published scientific papers and patents that disclose the conjugation of a cholesterol to drug molecules to improve delivery of the drug to cells (entry into cells/internalization), or improve solubility of the drug via micelle formation and subsequently the distribution when administered systemically.

PCT/EP2008/001188 and PCT/EP2010/001461 describe additional prior art which is relevant for defining the field of technology to which the present invention provides an enrichment.

In general, known controlled retention and release strategies are very cost-ineffective as they still require high doses of the drug and most of the drugs are turned-over into less active compounds before arriving at the target side. Moreover, high systemic concentrations often infer unwanted off-target effects of the drug, in particularly for chemotherapeutic compounds. Current drug carrier systems suffer from two major limitations: The drug is rapidly released from the carrier via a single burst release directly after applying the carrier-drug, and many drug-carrier systems do not allow local delivery to various sides in the human body using minimally invasive techniques. In fact, most drug release systems require surgical interventions or other invasive methods to get the carrier system in place, which is an additional burden for the patient.

In light of this, products, compositions, methods and uses that provide or improve the retention and release of agents, in particular agents that are (to be) provided to a subject, remain highly desirable, but are not yet readily available. In particular there is a clear need in the art for reliable, efficient and reproducible products, compositions, methods and uses that allow for better retention of such agents present in a carrier used to deliver the agent to the patient (i.e. that provide for prolonged retention of the agent in the carrier). Accordingly, the technical problem underlying the present invention can been seen in the provision of such products, compositions, methods and uses for complying with any of the aforementioned needs. The technical problem is solved by the embodiments characterized in the claims and herein below.

### DETAILED DESCRIPTION OF THE INVENTION

### Brief Description of the Drawings

Embodiments of the invention are further described hereinafter with reference to the accompanying drawings, in which:

These figures describe the synthetic route to modify Mitomycin C with a cholesterol and PEG motif; show sustained release from a UPy-PEG hydrogel under physiological conditions; reveal enhanced stability of the cholesterol-modified Mitomycin C; and demonstrate the biological activity of the drug after cholesterol modification; and demonstrate cytotoxicity after release from the hydrogel.

FIG. 1: Reaction scheme of Mitomycin-PEG₂₄-Cholesterol (MPC) from Mitomycin C (MMC), cholesterol and a PEG₂₄ linker. i DIPEA, CHCl₃, RT, O/N, 89%, ii NaOH, MeOH/H₂O, 50° C., O/N, quant, iii HATU/DIPEA, DMF, 50° C., O/N, 53%.

FIG. 2: UV absorption spectra of MMC (A) and MPC (B) at pH 7.4 and 4° C. Both MPC and MMC are stable.

FIG. 3: UV absorption spectra of MMC (A) and MPC (B) at pH 7.4 and 37° C. Both MPC and MMC are stable.

FIG. 4: UV absorption spectra of MMC (A) and MPC (B) at pH 6.5 and 37° C. MPC is stable over time whereas MMC degrades as indicated by changes in the UV absorption spectrum.

FIG. 5: Release from 10 wt % bifunctional UPy-PEG10k hydrogel. A: Release of MMC in approximately 24 hours from the hydrogel via diffusion kinetics. B: release of MPC from hydrogel in a controlled way for two weeks.

FIG. 6: Cytotoxicity (MTT) assay performed three times for a range of concentrations of MMC (A) and MPC (B) to CC531 cells. Both compounds have approximately 100% efficacy, but MPC exhibits a higher potency.

FIG. 7: Cytotoxicity after release from 10 wt % UPy-PEG1 Ok hydrogel loaded with nothing (blanco), MMC, MPC or a combination of MMC & MPC. Short term cytotoxicity due to burst release of MMC and long term cytotoxicity due to sustained release of MPC.

FIG. 8: Chemical structure of bifunctional UPy-PEG10k base polymer.

FIG. 9: Release of siRNA with or without the presence of an hydrophobic moiety, in particular cholesterol, from a 10 wt % UPy-PEG hydrogel.

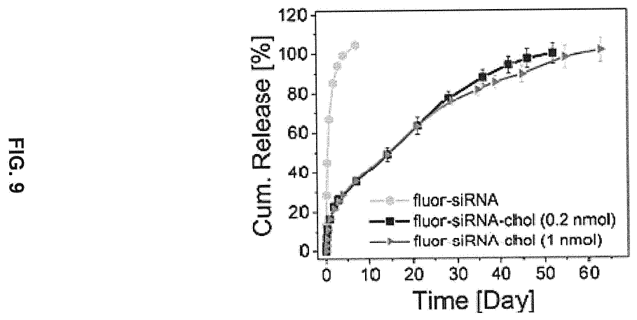

### DEFINITIONS

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

The description hereinafter of the claims specifying the exclusive rights on the present invention is deemed to be included in the description of this patent application. These exclusive rights cover also embodiments of the present invention not covered by the explicit wording of the claims but nevertheless forming obvious embodiments of the present invention for a person skilled in the art.

For purposes of the present invention, the following terms are defined below.

The term ‘agent’ as used herein refers to a compound that can be a drug, imaging moiety, agent or module, or other molecule that is suitable for application in the medical field or other fields where controlled retention and release of agents in chemical compositions are important.

The terms ‘compound’ and ‘molecule’ each refer, mutatis mutandis, to agent, as defined above.

The term ‘drug’ refers to a compound with a therapeutic effect and/or a drug function; the drug can be a small molecule drug, a chemotherapeutic drug, an anticancer drug, an anti-inflammatory drug, an anti-fibrotic drug, anti-infective drug, analgesic drug, an amino acid drug, a peptide/protein drug, hormone drug, a cosmetic additive, a nucleic acid based drug such as DNA, RNA, siRNA, miRNA or derivatives thereof. Within the context of the current invention such drug may be conjugated to a hydrophobic moiety as described herein such that the drug is provided with such (additional) hydrophobic moiety.

The term ‘carrier system’ or ‘carrier’ refers to a depot formed by a suitable chemical network, that is used to improve local delivery and effectiveness of an agent.

The term ‘hydrogel carrier’ refers to a carrier system in which the depot is formed by a network based on a hydrogel, which may suitably selected from the group consisting of UPy-PEG hydrogel, PEG-bisurea hydrogel, thermogels, pluronic F127, pNIPAM or any other suitable hydrogel comprising suitable hydrophobic domains.

‘Applicable chemical composition’ refers to a composition of the carrier system containing one or more agents in a form which renders the composition suitable for application in the medical field, including the field of cosmetics and diagnostics, e.g. medical imaging and other fields where controlled retention and release of agents in chemical compositions are important. In some embodiments, the applicable chemical composition comprises an agent that needs to be delivered to a subject, and wherein the agents has been provided with a hydrophobic moiety as described herein, and a carrier system that is used to deliver the agent that has been conjugated with the hydrophobic moiety.

The term ‘hydrophobic moiety or handle’ refers to a molecule such as cholesterol, and in some embodiments, to derivatives thereof with an hydrophobic character (log P value of cholesterol is about 7). Because of this hydrophobic character the molecule can interact with the network (carrier), for example, as described herein.

In some embodiment the hydrophobic moiety that is conjugated to the agent, for example to a pharmaceutical acceptable compound, is more hydrophobic than the compound with which it is conjugated, for example as determined by methods well known to the skilled person, for example using pH-metric and/or UV-metric method (see, for example, www.cvDrotex.com/product_sheets/Cyprotex_pKa_and_log_P_Product_Sheet.Ddf).

In some embodiment the agent that is conjugated to the hydrophobic moiety, for example to a cholesterol molecule or derivate thereof, is less hydrophobic than the hydrophobic moiety with which it is conjugated. As is understood by the skilled person hydrophobicity of a compound, agent or hydrophobic moiety may be described based on the log P value of the compound, agent, or hydrophobic moiety.

The term “log P value” as used herein refers to a measure of lipophilicity or hydrophobicity. Hydrophobicity tells about the compounds ability to dissolve into lipophilic (non-aqueous; non-polar) solutions (such as n-octanol) and/or in aqueous solution (such as water). The hydrophobic nature of a compound is typically measured as the compounds distribution between non-aqueous (n-octanol) and aqueous (water) phase and the result is expressed as a 10-base logarithm of the concentration ratios between these phases (partition coefficient), log P, e.g. as shown below for octano-water partition coefficient:

$$log {P}_{oct / wat} = log ( \frac{{[ solute ]}_{octanol}^{un \text{-} ionized}}{{[ solute ]}_{water}^{un \text{-} ionized}} )$$

In some embodiments, and in view of the invention disclosed herein, the conjugation of a hydrophobic moiety to the agent thus provides the agent with a moiety that is more hydrophobic than the initial compound.

In some embodiments, the hydrophobic moiety is cholesterol, or a derivate thereof, as described herein. In some embodiments, the agent that is conjugated with cholesterol or derivate thereof is less hydrophobic than cholesterol or derivate thereof.

In some embodiments, the term ‘cholesterol derivatives’ refers to aldosterone, beclomethasone, betamethasone, cholesterol, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, oxandrolone, oxymetholone, paramethasone, prednisolone, prednisone, stanozolol, and triamicinolone, testosterone, dehvdroeniandrosterone, androstenedione, dihydrotestosterone, aldosterone, estradiol, estrone, estriol, cortisol, oroaesterone and hydroxycholesterol.

Other suitable hydrophobic moieties include glycolipids, polycyclic aromatic hydrocarbon, phospholipids, saturated hydrocarbons, terpenoids, and unsaturated hydrocarbons.

The term ‘target site’ refers to the location in the human or animal body where the applicable chemical composition is administered to form a local depot.

The term ‘hydrophobic compartments’ refers to hydrophobic domains/pockets inside the carrier (e.g. inside the hydrogel network).

The term ‘suitable’ refers to what a person skilled in the art would consider technically required for the purpose, which is without undue burden technically feasible and for which no inventive effort or undue experimentation is required to arrive at./pct

‘OEG’ refers to oligo(ethylene glycol).

‘PEG’ refers to poly(ethylene glycol).

“UPy-PEG’ refers to the base polymer, comprising a suitable PEG-polymer functionalized (for example end-functionalized) with suitable ureido-pyrimidinone (UPy) moieties such as 2-ureido-4[1H]-pyrimidinone or its respective tautomer 2-ureido-4[3H]-pyrimidinone (both referred to as UPy), used to constitute the hydrogel network. Furthermore both the hydrogel (ureido-pyrimidinone (UPy)-poly(ethylene glycol) and the cholesterol motif are known. UPy-hydrogels are subject to various patent applications and is provided by, for example, SupraPolix B.V. For example EP1972661 discloses (and claims) the structure and preparation of hydrogels suitable a carrier for use in the current invention.

It describes a water gellant having the general structure (P)-(L)-(4H), where P represents the polymer backbone to which the 4H-unit (which, according to some embodiments, is UPy) is covalently connected via the hydrophobic linker L. The water gellant may have a molecular weight of 1200 to 1.000.000, preferably 2000 to 100.000, more preferably 3000 to 80.000, more preferably 5000 to 50.000 and most preferably 7.500 to 21.000 Dalton. The 4H-units can be attached to the polymer backbone P via the hydrophobic linker L in any way, e.g. by grafting onto the polymer backbone, by attachment to single or multiple—i.e. one, two or more—groups of the polymer backbone, or the 4H-units can be an integral part of the backbone of the polymer that constitutes the water gallant. For example, the polymer backbone P and the hydrophobic linker L may be connected via a (thio) urea; (Thio) urethane, amide, ester, carbonate, secondary amine, tertiary amine or ether moiety, for example the hydrophobic linker L and the 4H-unit are connected with a (thio) urea or a fraction amide. The polymer P may represent any type of polymer backbone known in the art, such as polyethers, polyesters, polyamides, polyacrylates, polymethacrylates, polyolefins, hydrogenated polyolefins, polysiloxanes, polycarbonates, (per)fluorinated polyethers, polyvinylenes, or co-polymers of such polymers. More preferably, the polymer backbone is a polyether, polyester, polyacrylate, polymethacrylate, polyolefin, hydrogenated polyolefin, polycarbonate, polyvinylene, or a co-polymer of such polymers. Even more preferred are polyethers, polyesters, or copolymers thereof. Most preferably, P is a polyether, preferably a polyglycol, preferably a polyethylene glycol or a poly ethylene-co-propylene glycol (random or block), most preferably a polyethylene glycol.

Also WO2016018145 describes suitable hydrogels for use in the current invention. The hydrogels are formed as by preparing a precursor solution by dissolving the water gellant (UPy-PEG) dissolved in water (buffer). In more detail, the liquid aqueous formulation comprises the water gellant dissolved in water. The amount of the water gellant in the liquid aqueous formulation ranges from about 0.3%-50.0% by weight, preferably from about 1%-25% by weight, more preferably from about 1%-20% by weight, more preferably from about 2%-10% by weight, and most preferably from about 2%-3% by weight, based on the total weight of the liquid aqueous formulation. The liquid aqueous formulation may contain additional functional ingredients that will contribute to the specific use of the hydrogel. Preferably the liquid aqueous formulation is prepared by dissolving the water gallant in water using basic pH, increased temperature or combination thereof. Preferably, the water contains a pH-buffer known in the art, such as a PBS or borate buffer. The cross-linked gel state is obtained by removing the external stimulus (high pH, high temperature) and switching the gel back to physiological conditions.

In particular EP1972661 discloses a hydrogel comprising:

(a) 0.3-50.0 wt. %, based on the total weight of the hydrogel, of a water gellant having the structure according to formula (A) or formula (B):

P-[L-(4H)]ₙ (A)

or

[P-L-(4H)-L-]ₚ (B)

wherein the water gellant is obtainable:
(i) by converting a polymer backbone P in a first step into a prepolymer according to the formula P-[L]ₙ, wherein in a second step the 4H-unit is introduced at the termini of the hydrophobic linker moiety L. wherein P and L are connected to each other by moieties selected from the group consisting of (thio)urea, (thio)urethane, amide, ester, carbonate, secondary amine, tertiary amine and ether moieties, and the 4H-unit and L are connected to each other by moieties selected from the group consisting of (thio)urea or amide moieties; or
(ii) by functionalizing a hydrophobic linker L in a first step at a first terminus thereof with a 4H-unit to provide a building block having the structure 4H-[L]q, wherein q represents the number of hydrophobic linker groups L attached to the 4H-unit and wherein q is I or 2, wherein the hydrophobic linker has a functional group at another terminus, and wherein in a second step the building block having the structure 4H-[L]_{q} is connected to the polymer backbone P, wherein P and L are connected to each other by moieties selected from the group consisting of (thio)urea, (thio)urethane, amide, ester, carbonate, secondary amine, tertiary amine and ether moieties, and the 4H-unit and L are connected to each other by moieties selected from the group consisting of (thio)urea or amide moieties; wherein:

n is in the range of 1.8 to 10;

p is in the range of 2 to 25;

L is a hydrophobic linker selected from the group consisting of cyclic, linear or branched C₂-C₂₄ alkylene groups, C₆-C₂₄ arylene groups,

C₇-C₂₄ alkarylene groups and C₇-C₂₄ arylalkylene groups, wherein the alkylene groups, arylene groups, alkarylene groups and arylalkylene groups optionally, but not preferably, comprise 1-5 heteroatoms selected from the group consisting of O, N and S;

the 4H-unit having the general formula (3) or (4), and tautomers thereof:

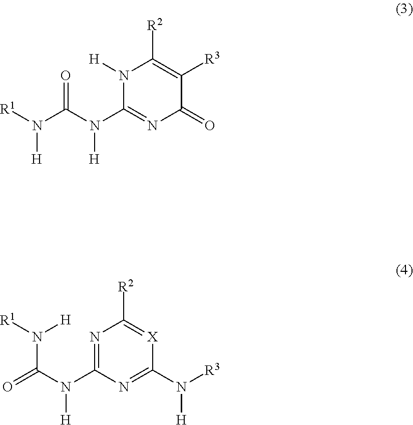

wherein X is nitrogen atom or a carbon atom bearing a substituent R¹⁵ and wherein R¹, R², R¹⁵ and R³ are independently selected from the group consisting of:
(1) hydrogen;
(2) C₁-C₂₀ alkyl;
(3) C₆-C₁₂ aryl;
(4) C₇-C₁₂ alkaryl;
(5) C₇-C₁₂ alkylaryl;
(6) polyester groups having the formula (5)

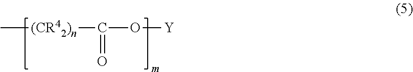

wherein R⁴ and Y are independently selected from the group consisting of hydrogen and C₁-C₆ linear or branched alkyl, n is 1-6 and m is 10 to 100;

(7) C, -C₁₀ alkyl groups substituted with 1-4 ureido groups according to the formula (6)

R⁵—NH—C(O)—NH— (6)

wherein R⁵ is selected from the group consisting of hydrogen and C₁-C₆ linear or branched alkyl;

(8) polyether groups having the formula (7)

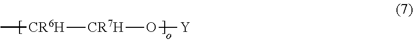

wherein Y, R⁶ and R⁷ are independently selected from the group consisting of hydrogen and C₁ C₆ linear or branched alkyl and o is 10-100; and wherein the 4H-unit is bonded to a polymer backbone via R₁, R₂, and/or R₃ (so that R₁, R₂ or R₃ represent a direct bond) with the other R groups representing, independently a side chain according to (1)-(8); and

(b) 50.0 to 99.7 wt. % water.

Suitable hydrogels are also described in, for example, EP2343342.

The term ‘linker’ refers to a (suitable) molecular spacer (e.g. oligomer or polymer) which provides a link between the agent and the suitable hydrophobic moiety (e.g. a drug and a cholesterol, respectively). In some embodiments, poly(ethylene glycol) (PEG) is preferably applied as linker in cholesterol (and derivate) modified (conjugated) agents. In some embodiments, oligo ethylene glycol (OEG) is preferably applied as linker in cholesterol (and derivate) modified (conjugated) agents. Other suitable linkers include but are not limited to oligosaccharides, oligopeptides, oligonucleotides, acrylates, acrylamides, polyvinyl alcohol, polyvinyl pyrrolidone, polyacrylic acid.

The term “controlled retention” or “controlling retention” or “enhanced controlled delivery” and the like as used herein is understood by the skilled person as all relating to modifying, in particular prolonging, the period in which an agent is present in the carrier. At the same time it relates to the rate of release of a compound from the carrier. In some embodiments, according to the invention, the agent conjugated with a hydrophobic moiety, is retained longer and is released more gradually and over an extended period of time from the carrier, in comparison to the same compound, but not conjugated with the hydrophobic moiety.

For the definition of other terms, not defined above or hereinafter, reference is made to published patent specifications and/or published scientific papers including theses, in which such terms have already been defined. These can without undue effort be found on the internet.

### DESCRIPTION OF THE INVENTION

All examples, preferences and embodiments described with respect to one aspect or embodiment of the invention is to be understood to be also explicitly described and disclosed with regards to any other aspect or embodiment of the invention as disclosed herein.

The present invention, providing a solution, relates to an applicable chemical composition containing an agent with a hydrophobic functional group (e.g. the agent has been provided with a hydrophobic moiety, such as cholesterol), e.g. a steroid, a sterol or in particular cholesterol (Log P value of 7), a derivative thereof, or any other chemical group that allows hydrophobic interactions, and possibly contains, but is not limited to, an additional OEG motif.

In some embodiment, the present invention, providing a solution, relates to an applicable chemical composition containing an agent, for example a drug, that is less hydrophobic than the hydrophobic moiety that is conjugated to the agent.

In some embodiments, the agent, e.g. drug, as a log P value (as defined herein) that is less than that of cholesterol (log P value of 7), preferably has a Log P values of 1.5 or less such as 1.4, 1.3, 1.2, 1.1, 1.0, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01, 0.00, −0.01, −0.02, −0.03, −0.04, −0.05, −0.06, −0.07, −0.08, −0.09, −1.0, −1.1, −1.2, −1.3, −1.4, −1.5, −1.6, −1.7, −1.8, −1.9, −2.0 or less)..

Examples of preferred compounds that van be conjugated with a hydrophobic moiety included, but are not limited to:

| | Log P value | Water solubility | |
|---|---|---|---|
| Gemcitabine: | −1.4 | 51.3 | mg/mL |
| Fluoroacil: | −0.9 | 11.1 | mg/mL |
| Mitomycin C: | −0.4 | 8.4 | mg/mL |
| Doxorubicin: | 1.27 | 1.18 | mg/mL |
| Daunorubicin: | 1.8 | 0.039 | mg/mL |
| Paclitaxel: | 3 | 0.00556 | mg/mL |

| | | |
|---|---|---|
| (Cholesterol: | 7 | Less than 0.000095 mg/mL) |

Other drugs that may be conjugated included of N-nitrosoureas, doxorubicin, daunorubicin, epirubicin, idarubicin, mitoxantrone, ametantrone, chlorambucil, bendamustine, melphalan, oxazaphosphorines, 5-fluorouracil, 2′-deoxy-5-fluorouridine, cytarabine, cladribine, fludarabine, pentostatine, gemcitabine, thioguanine, methotrexate, raltitrexed, pemetrexed, plevitrexed, paclitaxel, docetaxel, topotecan, irinotecan, 9-aminocamptothecin, camptothecin, vinblastine, vincristine, vindesine, vinorelbine, calicheamicins, maytansinoids, auristatins, epothilones, bleomycin, dactinomycin, plicamycin, mitomycin C and cis-configured platinum(II) complexes or any other suitable anticancer drug.

In other words, in some embodiments, the agent, e.g. drug, may also be an agent that has a higher water-solubility than the hydrophobic moiety, in particular than cholesterol.

In the context of the present invention, in some embodiments, said hydrophobic functional group or moiety is preferably cholesterol or a derivate thereof and as disclosed herein.

The modified compound (i.e. the agent that has been conjugated with a hydrophobic moiety) may be used alone or in combination with a carrier system, for example a hydrogel (e.g. UPy-PEG based hydrogel), for sustained release (e.g. controlled release) of the compound in the human body or in an aqueous environment.

In some embodiments, the agent conjugated with a hydrophobic moiety is preferably provided together with or is preferably present in a carrier system used for delivery of the agent to a subject.

To the knowledge of the inventors the combination of an agent conjugated with a hydrophobic moiety, in particular cholesterol or derivate thereof and a suitable carrier as described herein is currently not described or suggested in the art. The combination of both the agent conjugated with a hydrophobic moiety and the carrier, in particular a hydrogel, to create an (injectable) carrier system, e.g. hydrogel, with sustained release properties has not been described to the current knowledge of the inventors. What is also not described in the prior art is the advancement of the art as to the control of retention (diffusion) and thus the release of a drug molecule, e.g. an anticancer drug or an RNA/DNA compound, from a depot/reservoir (e.g a hydrogel such as the UPy-PEG hydrogel according to the present invention) by the conjugation of the drug to a hydrophobic moiety, which, according to a preferred embodiment of the present invention, is cholesterol but can also be another moiety with an hydrophobic character such as stearate or other. The inventors believe that it is the interaction between the drug-conjugate and the hydrogel depot that determines the remarkable retention/release/diffusion behavior.

Modification of the compound with a hydrophobic moiety, for example a cholesterol group or cholesterol-derivative is believed to improve the uptake of the compound by target cells and enhances the stability of the drug, thereby increasing the overall bioavailability of the compound at the side of delivery. Moreover, when used in combination with a carrier system that holds hydrophobic domains, the hydrophobic moiety, for example the cholesterol motif of the compound appears to prevent a sudden burst release, as is believed, via non-covalent interactions between the hydrophobic pockets in the carrier and the hydrophobic moiety, for example cholesterol motif, thereby improving the retention of the compound allowing for a sustained release over a longer period of time.

Thus it appears that modification of the compound with a hydrophobic moiety, e.g. with a cholesterol or derivate thereof, and as disclosed herein, increases the retention of the compound in a carrier system that preferably holds hydrophobic domains and increases the potency of the drug as it facilitates the cellular uptake and slows down the degradation of the drug once released. The improved retention of the compound allows for a sustained release of the active compound through a slow, gradual release of the compound when used in combination with this specific carrier system.

Thus, this combinatory compound-delivery system increases the therapeutic window for many drug molecules and requires a lower effective dose to induce the same therapeutic effect. This will likely be used by physicians in need for an alternative drug delivery system that benefit from a sustained release of the compound. Most likely this will be a physician in the area of oncology (oncologist, oncologic surgeons), endocrinology, cardiology and internal medicine. Also in the field of cosmetics and medical imaging the present delivery system may suitably be used.

Compounds that may suitably hydrophobically modified in accordance with the present invention (e.g. compounds or agents, that maybe conjugated with a hydrophobic moiety, preferably cholesterol, are for example imaging agents, therapeutic agents, bioactive molecules, proteins and peptides.

In some embodiments the agents, e.g. drugs, are agents that are less hydrophobic than cholesterol as defined by the Log P value, preferably having a Log P values of 1.5 or less, such as 1.4, 1.3, 1.2, 1.1, 1.0, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01, 0.00, −0.01, −0.02, −0.03, −0.04, −0.05, −0.06, −0.07, −0.08, −0.09, −1.0, −1.1, −1.2, −1.3, −1.4, −1.5, −1.6, −1.7, −1.8, −1.9, −2.0 or less. One non-limiting example of such compound is mitomycin C, which has a Log P value of −0.4 (as shown in the examples herein). Therefore, the results shown herein demonstrate that compounds, particularly compounds that are less hydrophobic than cholesterol, and which are modified with a hydrophobic moiety, in particular with cholesterol moiety, or derivate thereof, as taught herein, are retained and released from a carrier in a controlled, sustained manner.

In some embodiment the carrier is a hydrogel. The hydrogel that is preferably used to optimize this invention is based on UPy-PEG and ureido-pyrimidinone (UPy) has unique sol-gel characteristics. The system may be switched between solution and gel with temperature and with pH. For example, at a high pH the system is liquid but it forms a gel at physiological pH (pH 7.0-7.4). This characteristic allows the carrier to be administered locally and minimally invasive while in the liquid phase via injection with a catheter. While in the liquid phase drug molecules, or other functionalities, (conjugated with cholesterol or a derivate thereof) can conveniently be mixed in, which, after gelation of the gel, are then trapped inside the hydrogel and are released slowly. Fundamental to the dynamic behavior of such system is the supramolecular nature of the material, which allows for a modular material design via functionalization of potential interesting molecules with a UPy-moiety. Furthermore, due to the dynamic nature the UPy-PEG hydrogel is self-healing and can adapt physically towards its environment.

### EMBODIMENTS

In some embodiments, the inventions may be defined as follows:

1. Applicable chemical composition comprising an agent which has been conjugated to a hydrophobic moiety for locally controlling retention and release of the agent.

2. Applicable chemical composition according to embodiment 1 in which the agent is connected by a linker with the hydrophobic moiety.

3. Applicable chemical composition according to embodiment 1 or embodiment 2 in which the combination of a carrier system and the agent conjugated to or connected by a linker to a hydrophobic moiety is used for controlling the retention (diffusion) in the carrier at the target site or release of the agent from the carrier at the target site.

4. Applicable chemical composition according to embodiment 3 which comprises the agent and a suitable carrier system, which is a hydrogel comprising hydrophobic compartments/domains inside the hydrogel network, for enhanced controlled local delivery of the agent.

5. Applicable chemical composition according to embodiment 4 of which the carrier system is a macroscopic hydrogel carrier selected from the group consisting of UPy-PEG hydrogel, PEG-bisurea hydrogel, thermogels, pluronic F127, pNIPAM or any other suitable hydrogel comprising suitable hydrophobic domains

6. Applicable chemical composition according to embodiment 5 of which the macroscopic hydrogel carrier is UPy-PEG hydrogel.

7. Applicable chemical composition according to one or more of the preceding embodiments, in which the hydrophobic moiety is selected from the group consisting of steroids, sterols (cholesterol), polyaromatic hydrocarbons (pyrene), phospholipids, glycolipids, diacylglycerols, ceramides, saturated hydrocarbons, saturated fatty acids, unsaturated hydrocarbons, unsaturated fatty acids, isoprenoids (vitamin A and E), diamandoids (adamantane), hydrophobic peptides, hydrophobic proteins or is any other suitable moiety or suitable derivative with an hydrophobic character.

8. Applicable chemical composition according to embodiment 7, in which the hydrophobic moiety is cholesterol or a suitable cholesterol derivative.

9. Applicable chemical composition according to one or more of the preceding embodiments which is locally delivered by injection.

10. Applicable chemical composition according to one or more of the preceding embodiments in which the agent is a small molecule.

11. Applicable chemical composition according to one or more of the preceding embodiments in which the agent is a small molecule drug.

12. Applicable chemical composition according to one or more of the preceding embodiments in which the agent is a chemotherapeutic drug.

13. Applicable chemical composition according to one or more of the preceding embodiments in which the drug is an anticancer drug.

14. Applicable chemical composition according to embodiment 14 in which the anticancer drug is selected from the group consisting of N-nitrosoureas, doxorubicin, daunorubicin, epirubicin, idarubicin, mitoxantrone, ametantrone, chlorambucil, bendamustine, melphalan, oxazaphosphorines, 5-fluorouracil, 2′-deoxy-5-fluorouridine, cytarabine, cladribine, fludarabine, pentostatine, gemcitabine, thioguanine, methotrexate, raltitrexed, pemetrexed, plevitrexed, paclitaxel, docetaxel, topotecan, irinotecan, 9-aminocamptothecin, camptothecin, vinblastine, vincristine, vindesine, vinorelbine, calicheamicins, maytansinoids, auristatins, epothilones, bleomycin, dactinomycin, plicamycin, mitomycin C and cis-configured platinum(II) complexes or any other suitable anticancer drug.

15. Applicable chemical composition according to embodiment 14 in which the anticancer drug is Mitomycin C.

16. Applicable chemical composition according to one or more of the preceding embodiments in which the agent is an anti-inflammatory drug.

17. Applicable chemical composition according to one or more of the preceding embodiments in which the agent is a nucleic acid-based (DNA-/(si/mi)RNA-based) therapeutic compound or derivative thereof.

18. Applicable chemical composition according to one or more of the preceding embodiments in which the agent is an amino acid, peptide, or protein drug.

19. Applicable chemical composition according to one or more of the preceding embodiments in which the agent is an imaging agent for MRI, PET, SPECT or fluorescence spectroscopy.

20. Marketable product containing an applicable chemical composition according to one or more of the preceding embodiments

21. Process for the preparation of an applicable chemical composition or marketable product as embodiment in one or more of the preceding embodiments.

22. A composition as disclosed herein, comprising an agent, wherein said agent is conjugated to a hydrophobic moiety, and a carrier, preferably wherein:
said agent is a pharmaceutically acceptable compound, preferably a drug;
said hydrophobic moiety is a cholesterol or cholesterol derivate;
said carrier is a hydrogel comprising hydrophobic compartments, preferably the hydrogel is UPy-PEG hydrogel;

and wherein, preferably the agent is less hydrophobic than the hydrophobic moiety, even more preferably wherein the agent has a log P value of 1.5 or less, such as 1.4, 1.3, 1.2, 1.1, 1.0, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01, 0.00, −0.01, −0.02, −0.03, −0.04, −0.05, −0.06, −0.07, −0.08, −0.09, −1.0, −1.1, −1.2, −1.3, −1.4, −1.5, −1.6, −1.7, −1.8, −1.9, −2.0 or less.

23. The composition according to embodiment 22, wherein the agent is connected by a linker with the hydrophobic moiety, preferably wherein the linker is PEG or oligo ethylene glycol (OEG) or linkers such as oligosaccharides, oligopeptides, oligonucleotides, acrylates, acrylamides, polyvinyl alcohol, polyvinyl pyrrolidone, polyacrylic acid.

24. A method for modifying retention and/or release of a agent from a carrier, preferably an hydrogel comprising hydrophobic compartments, preferably the hydrogel is UPy-PEG hydrogel, the method comprising conjugation the agent to a hydrophobic moiety, preferably wherein the agent is less hydrophobic than the hydrophobic moiety, preferably wherein the hydrophobic moiety is cholesterol or a derivate thereof.

25. Agent, preferably an pharmaceutically acceptable compound for use in the treatment of a disease, wherein the agent that is administered to a subject in need thereof has been conjugated to a hydrophobic moiety, preferably a cholesterol or derivate thereof, and is present in a carrier, preferably an hydrogel comprising hydrophobic compartments, preferably the hydrogel is UPy-PEG hydrogel.

26, Method of treatment of a subject in need thereof, the method comprising treatment of the subject with an agent, preferably an pharmaceutically acceptable compound, wherein the agent has been conjugated to an hydrophobic moiety, preferably a cholesterol or derivate thereof, and is present in a carrier, preferably an hydrogel comprising hydrophobic compartments, preferably the hydrogel is UPy-PEG hydrogel.

Guidance of the Skilled Person

In order to successfully carry out the present invention the following is provided as guidance. The hydrogel should possess suitable specific characteristics (hydrophobic domains/pockets inside the hydrogel network) to allow favorable interactions and thus the retention/release profile as provided by the present invention. In other words not every hydrogel can be used. Therefore a suitable hydrogel composition should be used, i.e. a combination of drug and hydrogel that demonstrates a favorable release/retention profile. Hydrogels and hydrophobic moieties conjugated to desired drug molecules which hydrogels and hydrophobic moieties can be selected without an undue burden for the person skilled in the art, are covered by the present invention.

### EXAMPLES

All reagents and chemicals used in the examples were obtained from commercial sources at the highest purity available and used without further purification unless stated otherwise. Water was purified on an EMD Millipore Milli-Q Integral Water Purification System. Data processing and analysis was performed in Excel 2010 and Origin 2015. Where reported the standard deviation (SD) of the sample is given.

### Example 1. Preparation of Mitomycin-PEG-Cholesterol (MPC)

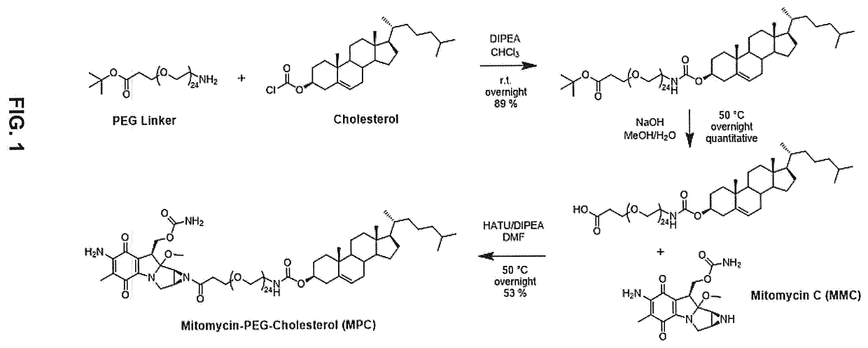

MPC was prepared in accordance with the reaction scheme specified in FIG. 1. Mitomycin C was purchased from Tocris (Cat. No. 3258). The PEG₂₄ linker was purchased from Iris Biotech. Cholesterol choloroformate and Thiazolyl Blue Tetrazolium Bromide was purchased from Sigma-Aldrich (M2128 SIGMA).

### Example 2. Stability Measurements by UV Absorption

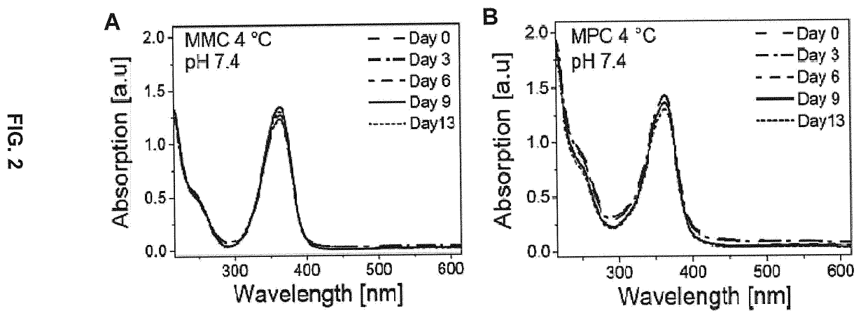

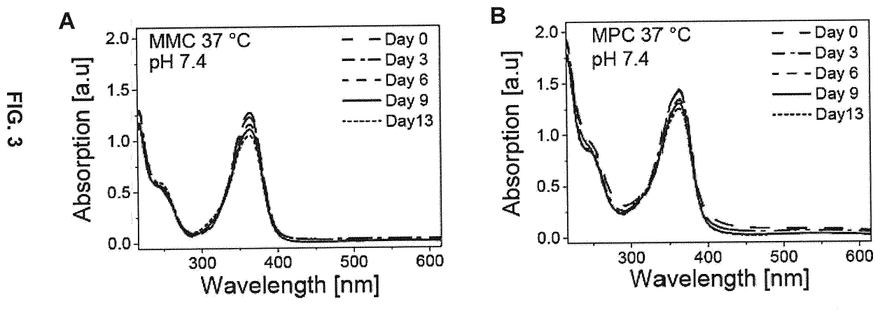

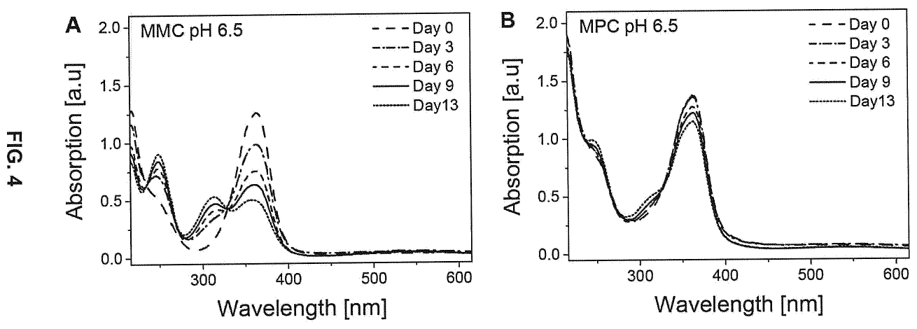

1 mg of MMC and MPC were taken from stock solutions (MMC, 2 mg/mL in MeOH; MPC, 4 mg/mL in CHCl₃) and dried in a rotary evaporator. Both were dissolved in 5 mL at a final concentration of 200 mg/L in PBS with some light warming and sonication. Samples were then split in three and incubated under different conditions. Condition 1: 4° C., pH 7.4; condition 2: 37° C., pH 7.4; condition 3: 37° C., pH 6.5. UV absorption was measured at day 0, 3, 6, 9 and 13 from 200-800 nm. UV absorption measurements were performed on a Varian Cary 50 Scan UV-Visible Spectrophotometer and Nanodrop ND 1000 Spectrophotometer. MMC and MPC were measured with a 1 mm and 1 cm path length respectively. For conditions 1 and 2, UV spectrometry showed no significant difference between the stability of MMC and MPC (FIGS. 2 and 3). This indicates, that at neutral pH, there is no significant difference in stability between these compounds, irrespective of temperature. However, at the slightly acidic pH of condition 3, MMC was unstable as opposed to MPC as shown in FIG. 4. It is known that at 37° C. and slightly acidic pH, such as in and around tumor tissue (pH ˜6.5), MMC is unstable and is rapidly degraded, which can also be seen in the absorption spectrum shown in FIG. 4A: The characteristic peak at 360 nm disappears, two new maxima appear at 317 and 250 nm and the minimum at 300 shifts to 275 nm. This degradation process is already severely evident after three days. In contrast hereto, FIG. 4B shows that MPC under the same conditions undergoes almost no degradation, over the course of two weeks. Part of the degradation process of MMC is opening of the aziridine-ring, which is most likely harder for MPC now that a PEG-Cholesterol is coupled to the ring. These results indicate that the functional group of MMC present in MPC is stabilized at the to-use temperature and pH.

### Example 3. Preparation of Bifunctional UPy-PEG10k Base Polymer

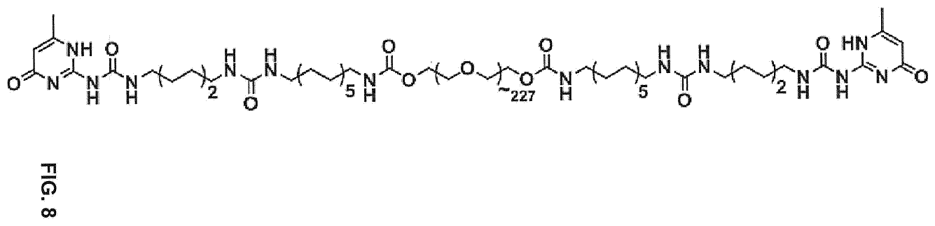

The hydrogel forming compound used in the examples is an UPy-PEG base polymer, wherein PEG has an Mn of 10 KDa corresponding to an average of 227 repeating units. This compound (a) is interchangeably referred to as “UPy-PEG10k base polymer” or “bifunctional UPy-PEG10k”. The chemical structure of the obtained bifunctional UPy-PEG10k is shown in FIG. 8. (2012) Hierarchical Formation of Supramolecular Transient Networks in Water: A Modular Injectable Delivery System. Adv. Mater., 24: 2703-2709. Synthesis is explained here for C10-hydrogels which is, mutatis mutandis, the same for C12 hydrogels

### Example 4. Preparation of Liquid Carrier System Composition Comprising MPC

The UPy-PEG10k liquid carrier composition was prepared by dissolving the UPy-PEG10k powder in PBS at elevated pH by stirring at 70° C. for 1 h using a magnetic stirrer (e.g. 10 mg in 90 μl PBS pH 11.7 for a 10 wt % hydrogel). Afterwards the viscous solution was cooled to RT with a resulting pH of approximately 9.0. MPC was added from 30 mM DMSO stock solution to final concentrations of 3 mM or 0.3 mM, resulting in a maximum of 10% DMSO in the final product. The obtained composition is a liquid composition at this pH.

### Example 5. Release Experiments

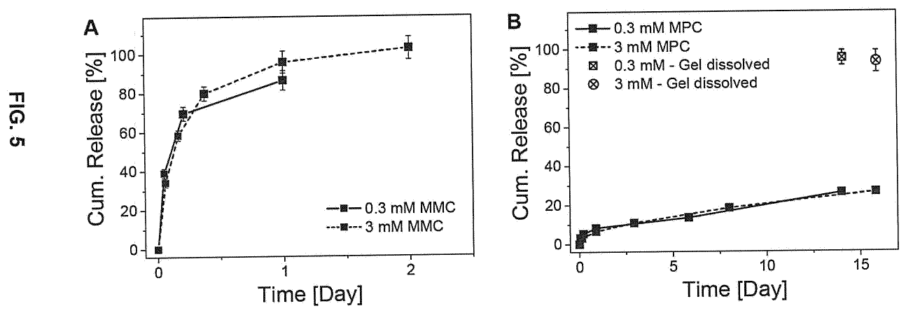

A liquid carrier composition comprising MPC was prepared as described in example 4. A liquid carrier composition comprising MMC was prepared in the same way by adding MMC to final concentrations of 3 mM or 0.3 mM instead of MPC. 100 μL of the solutions were transferred to Millicell hanging cell culture inserts (PIEP12R48/MCEP24H48) and immediately placed in a 24-well plate with 600 μL PBS per well. Contact with the neutral PBS through the membrane of the insert results in gelation of the liquid carrier, forming the hydrogel. At set time-points the PBS was refreshed and the removed PBS was analyzed for MMC or MPC content. Analysis was performed by measuring UV absorption and converted to concentration via predetermined standard curves. MPC gels were removed from the inserts after approximately two weeks by removing the membrane of the insert and gently pushing the still intact gel out. Gels were then dissolved in 1 mL basic PBS and after complete dissolution the concentration of MPC remaining in the gel was determined. The release from this 10 wt % bifunctional UPy-PEG10k hydrogel is shown in FIG. 5 and Table 1. MMC is released in approximately 24 hour from the hydrogel via diffusion kinetics (FIG. 5A). On the contrary, MPC is released from the hydrogel in a controlled way over two weeks (FIG. 5B). Release is independent of amount of incorporated MPC. High concentrations can be reached (0.3 mM=5.6 mg/mL). Release of MPC can be correlated to the erosion of the hydrogel. The initial goal to couple cholesterol to MMC to slow down the release from the hydrogel is accomplished hereby. MMC and MPC can potentially be combined in one treatment to obtain initial burst of MMC and subsequent sustained release of MPC.

TABLE 1

| MMC 0.3 mM | | MMC 3 mM | |
|---|---|---|---|
| Time(Day | Release (%) | Time(Day | Release (%) |
| 0.05 | 38.93 | 0.06 | 34.09 |
| 0.21 | 69.58 | 0.17 | 58.33 |
| 1.00 | 86.44 | 0.38 | 79.92 |
| | | 1.00 | 95.83 |
| | | 2.00 | 102.65 |
| MPC 0.3 mM | | MPC 3 mM | |
| Time(Day | Release (%) | Time(Day | Release (%) |
| 0.04 | 3.28 | 0.13 | 3.20 |
| 0.21 | 5.35 | 0.88 | 6.63 |
| 0.88 | 8.33 | 2.92 | 10.84 |
| 5.83 | 13.43 | 8.00 | 18.31 |
| 14.00 | 26.12 | 15.83 | 26.41 |
| Gel Dissolved | 95.21 | Gel Dissolved | 93.33 |

### Example 6. MTT Assays

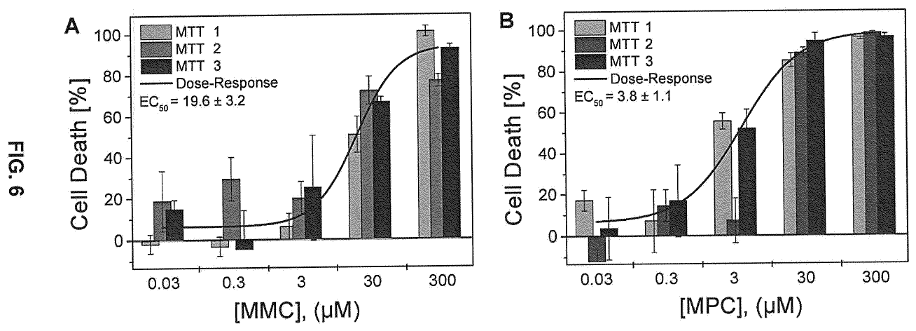

For viability assays 15 k cells were seeded in a 96 well plate 24 hours prior to the experiment. MMC and MPC were taken from organic solvent stock solutions, dried in rotary evaporator and dissolved in fully supplemented cell medium to the desired concentrations. Seed medium was discarded and replaced by 170 μL of the medium containing MMC or MPC. After approximately 24 hour incubation in the cell incubator, 25 μL of freshly prepared MTT solution (5 mg/mL in PBS, filtered through 0.2 μm filter) was added to the wells and incubated for 2 hours. The medium was then completely removed and replaced with 125 μL 0.04 M HCl in isopropanol and incubated for 1 hour. After gently mixing, 100 μl of the isopropanol solution was transferred to a Costar EIA/RIA 96 transparent flat bottom wells plate. The absorbance was measured at 570 nm with 650 nm as reference. Absorbance of MTT was read out on a Tecan Saffire 2 plate reader and Tecan Spark 10M plate reader. Values were normalized to untreated cells. The complete assay was performed three times for CC531 cells. All data sets were used together to fit a global sigmoidal dose response curve with a shared slope and center, giving one EC₅₀ per cell line. A concatenate fit was plotted through the MTT results for visualization. FIG. 6 shows the results of the MTT assay performed three times for a range of concentrations of MMC (A) and MPC (B) to CC531 cells. Both compounds have approximately 100% efficacy, but MPC exhibits a higher potency. Fitting with a sigmoidal dose response curve function gives EC₅₀ of 19.6±3.2 μM and 3.8±1.1 μM for MMC and MPC, respectively. Every experiment n=8, ±SD.

### Example 7. Release and Toxicity

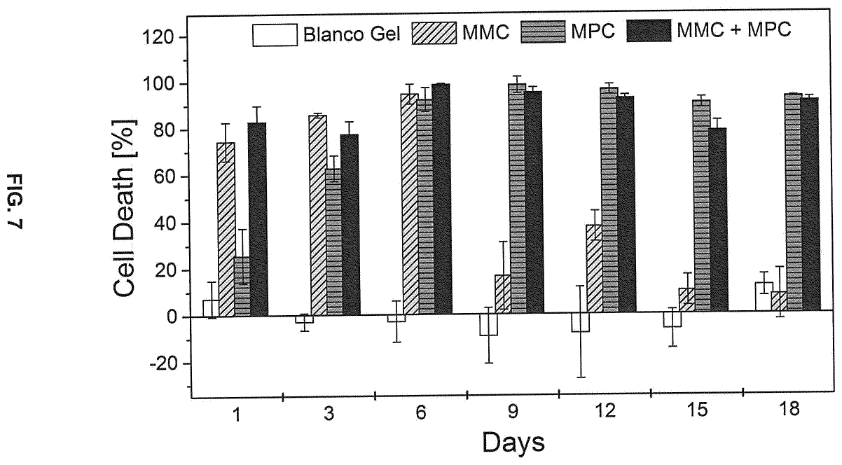

Release and toxicity were combined by preparing gels containing no drug (blanco), MMC, MPC or half MMC & half MPC at total 1.8 mM concentrations in Millicell hanging cell culture inserts (PIEP12R48/MCEP24H48) that were placed directly above 70 k cells CC531 cells seeded in a 24 wells plate in 600 μL medium. At each time-point the gels were transferred to freshly seeded cells and MTT assays were performed on the cells that were incubated with the gels in inserts. MTT assays were performed as described previously but now with 100 μL MTT solution and 400 μL 0.04 M HCl in isopropanol. The results are shown in FIG. 7. As expected the MMC loaded hydrogel immediately shows a high activity towards killing the cells, since most of the MMC compound is quickly released. However, after a few days the effect is diminished since all material has been released. For MPC the effect is much longer, at least for the duration of the experiment (18 days) due to sustained release. However there is barely an effect in the first day because there is no burst release present. When the two compounds are combined there is high cell death for the complete duration of the experiment, due to the burst release of MMC followed by the sustained release thereafter of MPC.

1. Applicable chemical composition comprising an agent which has been conjugated to a hydrophobic moiety for locally controlling retention and release of the agent.

2. Applicable chemical composition according to claim 1 in which the agent is connected by a linker with the hydrophobic moiety.

3. Applicable chemical composition according to claim 1 in which the combination of a carrier system and the agent conjugated to or connected by a linker to a hydrophobic moiety is used for controlling the retention (diffusion) in the carrier at the target site or release of the agent from the carrier at the target site.

4. Applicable chemical composition according to claim 3 which comprises the agent and a suitable carrier system, which is a hydrogel comprising hydrophobic compartments/domains inside the hydrogel network, for enhanced controlled local delivery of the agent.

5. Applicable chemical composition according to claim 4 of which the carrier system is a macroscopic hydrogel carrier selected from the group consisting of UPy-PEG hydrogel, PEG-bisurea hydrogel, thermogels, pluronic F127, pNIPAM or any other suitable hydrogel comprising suitable hydrophobic domains

6. Applicable chemical composition according to claim 5 of which the macroscopic hydrogel carrier is UPy-PEG hydrogel.

7. Applicable chemical composition according to claim 1, in which the hydrophobic moiety is selected from the group consisting of steroids, sterols (cholesterol), polyaromatic hydrocarbons (pyrene), phospholipids, glycolipids, diacylglycerols, ceramides, saturated hydrocarbons, saturated fatty acids, unsaturated hydrocarbons, unsaturated fatty acids, isoprenoids (vitamin A and E), diamandoids (adamantane), hydrophobic peptides, hydrophobic proteins or is any other suitable moiety or suitable derivative with an hydrophobic character.

8. Applicable chemical composition according to claim 7, in which the hydrophobic moiety is cholesterol or a suitable cholesterol derivative.

9. Applicable chemical composition according to claim 1 which is locally delivered by injection.

10. Applicable chemical composition according to claim 1 in which the agent is a small molecule.

11. Applicable chemical composition according to claim 1 in which the agent is a small molecule drug.

12. Applicable chemical composition according to claim 1 in which the agent is a chemotherapeutic drug.

13. Applicable chemical composition according to claim 1 in which the drug is an anticancer drug.

14. Applicable chemical composition according to claim 14 in which the anticancer drug is selected from the group consisting of N-nitrosoureas, doxorubicin, daunorubicin, epirubicin, idarubicin, mitoxantrone, ametantrone, chlorambucil, bendamustine, melphalan, oxazaphosphorines, 5-fluorouracil, 2′-deoxy-5-fluorouridine, cytarabine, cladribine, fludarabine, pentostatine, gemcitabine, thioguanine, methotrexate, raltitrexed, pemetrexed, plevitrexed, paclitaxel, docetaxel, topotecan, irinotecan, 9-aminocamptothecin, camptothecin, vinblastine, vincristine, vindesine, vinorelbine, calicheamicins, maytansinoids, auristatins, epothilones, bleomycin, dactinomycin, plicamycin, mitomycin C and cis-configured platinum(II) complexes or any other suitable anticancer drug.

15. Applicable chemical composition according to claim 14 in which the anticancer drug is Mitomycin C.

16. Applicable chemical composition according to claim 1 in which the agent is an anti-inflammatory drug.

17. Applicable chemical composition according to claim 1 in which the agent is a nucleic acid-based (DNA-/(si/mi)RNA-based) therapeutic compound or derivative thereof.

18. Applicable chemical composition according to claim 1 in which the agent is an amino acid, peptide, or protein drug.

19. Applicable chemical composition according to claim 1 in which the agent is an imaging agent for MRI, PET, SPECT or fluorescence spectroscopy.

20. Marketable product containing an applicable chemical composition according to claim 1.

21. Process for the preparation of an applicable chemical composition according to claim 1.
